(19) United States
(12) Patent Application Publication
(10) Pub. No.: US 2018/0011094 A1
(43) Pub. Date: Jan. 11, 2018

(54) LPL/RLP FOR ASSESSMENT OF CARDIOVASCULAR DISEASE RISK

(71) Applicant: TRUE HEALTH DIAGNOSTICS, LLC, Frisco, TX (US)

(72) Inventors: Dan Hoefner, Frisco, TX (US); Russ Warnick, Frisco, TX (US)

(21) Appl. No.: 15/544,103

(22) PCT Filed: Jan. 21, 2016
(86) PCT No.: PCT/US16/14345

§ 371 Date: Jul. 17, 2017

Related U.S. Application Data
(60) Provisional application No. 62/105,958, filed on Jan. 21, 2015.

(51) Int. Cl. G01N 33/573; G01N 33/92

(52) U.S. Cl. CPC G01N 33/573; G01N 33/92; C12Y 301/01034; G01N 2800/50; G01N 2800/32

(57) ABSTRACT

In various embodiments, the application relates to methods for assessment of cardiovascular disease (CVD) risk in a patient comprising comparing pre-heparin LPL (lipoprotein lipase) levels which are associated with protection from CVD to remnant lipoprotein cholesterol (RLP-C) and/or remnant lipoprotein triglyceride content (RLP-Tg).

### PRIORITY CLAIM

This application is a 371 U.S. National Stage Application of International PCT Application No. PCT/US16/14345 filed on Jan. 21, 2016, which claims priority to U.S. Provisional Application Ser. No. 62/105,958 filed Jan. 21, 2015, the entire contents of which are incorporated by herein reference and relied upon.

### TECHNICAL FIELD

In various embodiments, the present invention involves the assessment of cardiovascular disease (CVD) risk in a patient by comparing the pre-heparin LPL (lipoprotein lipase) levels which are associated with protection from CVD to the remnant lipoprotein cholesterol (RLP-C) and/or the remnant lipoprotein triglyceride content (RLP-Tg), which contribute to CVD development and progression.

### BACKGROUND

Blood contains various kinds of lipoproteins, including chylomicron (CM), very low-density lipoprotein (VLDL), low-density lipoprotein (LDL), high-density lipoprotein (HDL), and the remnants of CM and VLDL. Traditionally, HDL-cholesterol has been considered as a negative risk factor for arteriosclerosis and LDL-cholesterol has been considered as a positive risk factor for arteriosclerosis. Recently, there is increased focus on predicting the risk of coronary artery disease (CAD) by measuring serum triglyceride (TG) levels, independent from total cholesterol, LDL-C, and HDL cholesterol^{1, 2, 3}. Serum TG-rich lipoproteins (TRL) comprise apoB-48-carrying chylomicrons of intestinal origin and apoB-100-carrying VLDL of hepatic origin together with the remnant-like lipoproteins (RLPs) of both classes. Increased levels of non-fasting TG levels strongly correlate with an increase in RLPs, and is associated with an increased risk of myocardial infarction (MI), ischemic heart disease (IHD), and total death in men and women in the general population^{2, 4}. Indeed, individuals with type 2 diabetes have increased levels of remnant lipoproteins that contribute to increased atherogenicity^{4, 5}.

RLP-cholesterol (RLP-C), a component of TRL, is found to be significantly higher in patients with CAD^{6, 7} and an independent CAD risk factor for women in the fasting state⁸. Another fraction of TRL, RLP-triglyceride (RLP-Tg) is also a strong pathologic factor in sudden cardiac death cases⁹. RLPs are thus useful diagnostic markers for predicting atherogenicity.

A method for the isolation and quantification of plasma remnants was first developed by Nakajima et al (Jimro II, Otsuka Pharmaceutical, Japan); this is an immunoadsorption method, and RLP is separated from serum by immunoaffinity chromatography using affinity gel containing anti-apoA-I and anti-apoB-100 monoclonal antibodies, and cholesterol contained in the separated RLP is determined. Kyowa Medex Co (U.S. Pat. No. 8,119,360B2) developed a method for measuring RLP-C without the need for separation of the components of a sample; the sample is treated with cholesterol esterase and cholesterol oxidase that specifically act on RLP-C to convert esterified cholesterol to free cholesterol. This free cholesterol is acted upon by cholesterol oxidase and the resultant hydrogen peroxide is then measured to correlate to the amount of RLP-C present in the sample. Otsuka Pharmaceuticals (U.S. Pat. No. 7,799,537B2) developed a method on a similar principle, but with a cholesterol esterase in which the activity ratio of lipoprotein lipase to cholesterol esterase is high, such that the sensitivity to measure cholesterol in RLP is higher than that offered by other methods. Daichii Pharmaceuticals developed a method wherein the sample is treated with a cholesterol esterase having a molecular weight of more than 40 kDa and not having a subunit having a molecular weight of not more than 40 kDa. RLP-Tg has been measured by a modified version of the commercially available Determiner LTGII kit (Kyowa Medex Co. Ltd, Tokyo) and TG-EX (Denka Seiken Co. Tokyo). RLP-Tg is also measured by an enzymatic method wherein the sample is treated by a surfactant that specifically helps release glycerol from a triglyceride containing lipoprotein, which is then acted upon by lipoprotein lipase to release hydrogen peroxide that is indicative of the amount of RLP-Tg in the sample. Hence, currently, commercialized kits designed for measuring LDL-Tg have been used for measuring RLP-Tg.

Lipoprotein lipase (LPL) plays a vital role in lipoprotein metabolism by catalyzing the hydrolysis of TGs in chylomicrons and VLDL particles and is regulated by insulin^{10, 11}. Pre-heparin LPL mass positively correlates with HDL-C and negatively with VLDL-TG in men with coronary heart disease,^{12, 13, 14}. Interestingly, in patients with increased RLPs and hyperlipidemia, pre-heparin LPL mass levels were lower than that in control patients,¹⁵. Hence, the levels of preheparin LPL are inversely correlated with the RLP levels.

Moreover, it was recently reported that in young women, serum adiponectin is positively associated with HDL-C and serum LPL and inversely with body mass index and triglyceride levels,¹⁶. Considering adiponectin plays a major role in TG metabolism and that LPL levels correlate with adiponectin levels even in normal patients,¹⁷ it is clear that adiponectin and LPL levels are indicative of the risk of CVD.

It is an object of the present invention to provide a new technique for accurately assessing the risk factors for CVD. The invention is based upon the finding that low levels of preheparin LPL is inversely proportional to RLPs indicating high CVD risk.

### DESCRIPTION

In various embodiments, the instant disclosure relates to the measurement of lipoprotein lipase (LPL) and the remnant-like lipoproteins (RLP), RLP-cholesterol (RLP-C) or RLP-triglyceride (RLP-Tg), wherein the LPL/RLP ratio correlates to the risk of cardiovascular disease. In one embodiment, the test comprises measuring LPL by ELISA and or use a reliable automated kit for pre-heparin LPL. In another embodiment, the disclosure provides methods using an assay to measure RLP-C, or a pretreatment of RLP-Tg and an automated analysis of RLP-Tg.

In one embodiment, the invention provides a ratio for converting the inputs of LPL and an RLP species to an independent score for determination of cardiovascular disease risk: generally, the good component LPL and the bad carrier are compared by a normalized ratio against a standard population result for the ratio. The ratio may be normalized to a score itself, or compared to the ratios from a population and determined as a score after the comparison.

We have evidence that LPL is critical to the metabolic pathway for remnants. Pre-heparin LPL is associated with protection from CVD, as is adiponectin and there is emerging evidence that adiponectin regulates LPL.

There is abundant evidence that remnants (RLP-C) contribute to CVD and the remnant trig content (RLP-Tg) has been shown to be a stronger predictor than the cholesterol content of RLP; hence the rationale for considering both measures of remnants.

In one embodiment, the invention provides a combination of these markers in a statistically-normalized ratio to provide a useful prediction of the development and progression of patients at risk for or suffering from cardiovascular disease.

In one aspect the invention provides a method of measuring cardiovascular disease risk in a patient. A sample is typically first obtained from a patient. Generally the patient is fasting at the time of measurement. The patient may provide a sample individually through a blood or tissue sample. In some cases, a health care provider may obtain a blood or tissue sample from a patient. The sample may be processed into plasma, serum, or a sample of spinal cord, or cerebrospinal fluid may be provided. The sample may be used at a local laboratory, and central reference laboratory, at the bedside, or with any analysis system remote or in proximity to the patient.

Analysis systems may include any apparatus for the processing and analysis of samples. For example, an enzyme-linked immunosorbent assay (ELISA)-based apparatus may be a high-throughput system for analysis of multiple samples in parallel. It can also, for example, be a point-of-care or distributed system. It may be on a microchip, microfluidic system, a parallel-flow system or other means for performing an immunoassay.

LPL may be measured from a patient sample, including from plasma, using an immunoassay such as an ELISA. Specifically, pre-heparin plasma LPL mass is quantified through the ELISA format. An expected range for pre-heparin LPL is up to about 60 ng/ml, with diseased patients typically having a low LPL mass of 15 ng/ml and below. Reduced levels of serum LPL are associated with premature atherosclerosis and an increased risk of developing future coronary artery disease. The preheparin LPL mass may be lower, around 40 mg/dL in patients with known disease. For example, preheparing LPL was found to be 41 mg/dL in patients with acute myocardial infarction, and about 38 ng/mL in patients with coronary atherosclerosis. Additionally, LPL activity positively correlates with adiponectin and HDL-C and inversely with triglyceride levels and body weight.

RLP-c or RLP-Tg may be measured by a variety of methods, some involving immunoadsorption or enzymatic assay. RLP-C and RLP-Tg levels are significantly elevated in patients with coronary heart disease. The expected range for RLP-Tg in patients is under about 50 mg/dL, found at 49 mg/dL in a recent study, and greater than about 80 mg/dL in patients with serious CVD events like sudden cardiac death events, found at 81 mg/dL in a recent study. RLP-c is generally found in concentrations of under about 60 mg/dl for normal patients and unhealthy patients with greater levels than about 6 mg/dl. RLP-c is generally found in concentrations of about 5.7 mg/dl in normal patients and greater than 7.6 mg/dL in patients with coronary artery disease having >50% occlusion in at least one major vessel. In patients with type III dyslipidemia who are at very high risk of CVD, RLP-c levels were markedly elevated at about 31-240 mg/dL. However, in spite of the marked changed in RLP and LPL levels in diseased patients, the range of values correlating with healthy and unhealthy patients has been shown to be inconsistent and the ratio of LPL to RLP-c or RLP-tg is a better predictor of disease risk. While there is data indicating that elevated RLP levels and reduced LPL activity are independent risk factors for developing cardiovascular disease, each risk factor on its own cannot be considered conclusive. It has been found that individuals having high LPL and low RLP levels within the normal range may be at risk of developing cardiovascular disease. For this reason, a more reliable risk factor is required and a ratio of LPL to RLP-c or RLP-Tg is a better predictor of disease risk and provides critical information on the health of an individual.

In one embodiment, calculating a score from the measured levels of LPL and either RLP-c or RLP-Tg involves a linear combination of logarithmic transformations of each concentration. Such a calculation follows the formula: A=B*ln([LPL])−C*ln([RLP-c or RLP-tg]), where B and C are coefficients calculated from the correlation of values to a population distribution. In some cases, the score may be calculated without logarithmic transformations, wherein the formula is A=B*[LPL]/C*([RLP-c or RLP-tg]), or some derivation thereof. The ratio of LPL/RLP in an individual is compared to that of a normal control in order to correlate the risk of that individual having CVD or other diseases. Preferably, the level of preheparin LPL in a normal control is not less than 60 mg/dL. Preferably, the level of RLP-c is not more than 6 mg/dL and that of RLP-Tg is not more than 50 mg/dL.

Although the ratio of LPL/RLP of an individual compared to that of a normal control provides valuable information as to the general health, this information may also be taken in conjunction with other biological variables, including additional factors such as adiponectin, total serum cholesterol, and HDL-C concentrations. Additionally, the concentration may be substituted by an activity measurement.

The calculated score may be compared to scores from a population of other patients, placing the patient on a spectrum of cardiovascular risk. For example, a calculated ratio that ranks above the 50^{th}, 75^{th}, 90^{th}, 95^{th}, 99^{th}, or other percentile in the distribution of ratios from a population may be assigned a high risk level. It has been found that there is a positive correlation between the preheparin LPL level (and LPL/RLP ratio of 1 or above) and a favorable cholesterol profile and a positive correlation between the RLP levels (and LPL/RLP ratio of below 1) and a poor cholesterol profile.

In one embodiment, the score generated by the algorithm is an odds ratio that corresponds to the likelihood that a patient will develop cardiovascular disease, progress into more advanced cardiovascular disease, or suffer a cardiac event. The odds ratio is a measure of relative risk determined by logistic regression. The interpretation is that for every increase of the algorithm score of 1 SD, the odds of risk for the disease development, progression, or event increase by a given amount. Generally, an individual having a high LPL mass and low RLP levels compared to a normal control requires no therapeutic intervention and will be termed “low risk”. An individual with RLP-c values higher than 7.6 mg/dL but lower than 30 mg/dL, RLP-Tg values higher than 50 mg/dL but lower than 70 mg/dL, and LPL levels lower than 40 mg/dL would be assigned a risk level of “intermediate” and should be assessed further for lipid metabolic disorders. An individual with RLP-c values higher than 30 mg/dL, RLP-Tg values higher than 70 mg/dL, and LPL levels lower than 30 mg/dL would be assigned a “high” risk level and is at a risk of CVD.

The score and/or risk level is reported the risk level to the patient or patient's health care provider. The information may be documented on tangible form, such as a paper report which is mailed or faxed to the patient or the patient's healthcare provider. Alternately, the results may be transferred via electronic means and viewed via a secure internet-connected portal, such as a website or mobile application. The results may be presented with other test results in a panel. The results may additionally be presented with suggestions for treatment or lifestyle changes to improve the patient's health outcomes.

The following references are incorporated by reference herein, in their entirety:
1) H. Iso, Y. Naito, S. Sato et al (2001)
2) S. Bansal et al (2007)
3) B. G. Nordestgaard (2007)
4) Twickler T B Circulation (2004)
5) Chapman M J Eur Heart J (2011)
6) S. Devraj et al Am J Med (1998)
7) K. Kugiyama et al Circulation (1999)
8) J. R. McNamara et al Atherosclerosis (2001)
9) Nakajima et al Atherosclerosis (2008)
10) Havel R J J Clin Invest 1973
11) Vydelingum N Am J Physiol 1983
12) Tornvall P Arterioscler Thromb Vasc Biol 1995
13) Tornvall P Metabolism 1996
14) Kobayashi J Metab Res 2001
15) Watanabe H Atherosclerosis 1999
16) Terazawa-Watanabe M, Tsuboi A, Fukuo K, Kazumi T. Metab Syndr Relat Disord. 2014
17) Liping Qiao et al Diabetes 2008
16) US20030207342 (7202047) A1 Kyowa Medix: Method and reagent for determination of cholesterol in remnant like particles
A method for the quantitative determination of cholesterol in remnant-like particles in a biological sample, which comprises contacting the biological sample with (i) cholesterol esterase, (ii) cholesterol oxidase or cholesterol dehydrogenase, and (iii) phospholipase in an aqueous medium in the presence of oxygen or an oxidized coenzyme, and measuring the formed hydrogen peroxide or reduced coenzyme.
17) US20090023167 A1 Kyowa Medix (8119360): Method, reagent and kit for determination of cholesterol in remnant-like particles (rlp)
A method for quantitatively determining remnant-like particle cholesterol in a sample (involves the use of particular polymeric matrices for the separation of cholesterol)
18) US2013230873A1 Denka Seiken: Method For Quantification Of Remnant-Like Lipoprotein Cholesterol And Kit For Same
A method for quantifying cholesterol in a remnant-like lipoprotein in a sample containing different lipoproteins including the remnant-like lipoprotein (without the need of separation) comprises a step (1) of erasing cholesterol in lipoproteins other than the remnant-like lipoprotein (by a cholesterol esterase of molecular weight >40 kDa); and a step (2) of quantifying cholesterol in the remaining remnant-like lipoprotein.
19) U.S. Pat. No. 7,799,537B2 Jimro Co. Ltd (Otsuka Pharmaceutical): Cholesterol measuring reagent containing a cholesterol esterase
A method for measuring cholesterol in remnant-like lipoprotein, comprising measuring cholesterol in the lipoprotein by measuring hydrogen peroxide or a reduced coenzyme obtained by allowing a cholesterol esterase and a cholesterol oxidase or a cholesterol dehydrogenase to act on a test sample containing a lipoprotein, wherein said cholesterol esterase has lipoprotein lipase activity and cholesterol esterase activity wherein the activity ratio of lipoprotein lipase activity to cholesterol esterase activity ranges from 12:1 to 7000:1.
20) U.S. Pat. No. 6,811,994B1 Kyowa Medix: Method for quantitating triglycerides in lipoproteins A method for quantitating triglyceride in a particular lipoprotein in a sample containing triglycerides in a mixture of lipoproteins and free glycerol which comprises the steps of: (1) eliminating the free glycerol from the sample, (2) reacting the sample from step (1) which contains the mixture of the lipoprotein with lipoprotein lipase to produce glycerol in the presence of a reagent which inhibits a reaction of lipoproteins with the lipoprotein lipase other than the particular lipoprotein, (3) reacting the sample from step (2) with an enzyme system which generates hydrogen peroxide from free glycerol, and (4) quantitating generated hydrogen peroxide from step (3),wherein the particular lipoprotein is high density lipoprotein.
21) U.S. Pat. No. 7,335,483B2 Daiichi Pure Chemicals Co., Ltd.: Bioassay component for use in determining concentration of lipids in blood; diagnosing cardiovascular disorders
A quantitation kit for cholesterol in a specific lipoprotein, comprising (1) a first reagent comprising (a) cholesterol oxidase, (b) a reaction accelerator selected from flufenamic acid, mefenamic acid, 2,2′,6′,2″-terpyridine, tiglic acid, fusidic acid, betamethasone acetate, monensin or mevinolin, and (c) a hydrogen peroxide consuming substance; and (2) a second reagent comprising a substance which acts upon said specific lipoprotein only, cholesterol esterase, and a color developer.

1. A method of assessing a level or severity of cardiovascular disease risk in a subject comprising:
(a) measuring a level of a species of lipoprotein lipase (LPL) in a biological sample from the subject;
(b) measuring a level of remnant lipoprotein cholesterol (RLP-c) and/or remnant lipoprotein triglyceride (RLP-Tg) in the biological sample;
(c) transforming the measured levels in steps (a) and (b) to a ratio or logarithmic score based on any of the following models:
I. ratio score=LPL/RLP-c;
II. ratio score=LPL/RLP-Tg; or
III. logarithmic score=B*log([LPL])−C*log([RLP-c or RLP-tg]), wherein B and C are coefficients calculated from the correlation of values to a population distribution;
(d) comparing the score obtained in step (c) to a reference score; and
(e) assessing the cardiovascular disease risk of the subject based on the said comparison; wherein an increased, decreased or unchanged score relative to the reference score indicates the level or severity of the cardiovascular disease risk in the subject.

2. The method of claim 1, wherein a score higher than the reference score indicates an increased risk of cardiovascular disease in the subject.

3. The method of claim 1, wherein the species of LPL is a pre-heparin LPL.

4. The method of claim 1, wherein the LPL species is measured by immunoassay.

5. The method of claim 1, wherein the LPL species is measured by ELISA.

6. The method of claim 1, wherein RLP-c is measured by an enzymatic assay or by immunoadsorption.

7. (canceled)

8. The method of claim 1, wherein RLP-Tg is measured by an enzymatic assay or by immunoadsorption.

9. (canceled)

10. The method of claim 1, wherein the levels of adiponectin are additionally measured in the biological sample by ELISA.

11. The method of claim 1, wherein step (d) further comprises assigning a risk level to the subject based on the comparison.

12. The method of claim 11, wherein the risk level comprises low, medium or high categories representing a low, medium or high risk, respectively, of developing cardiovascular disease, for progression of cardiovascular disease, or for having a cardiac event.

13-14. (canceled)

15. The method of claim 1, wherein the subject suffers from cardiovascular disease (CVD).

16. The method of claim 1, wherein the reference score is obtained by any of models I-III from a population comprising subjects suffering from CVD, healthy individuals, or subjects suffering from a metabolic disease, diabetes or a diabetes-related condition.

17-18. (canceled)

19. The method according of claim 1, wherein any of models I-III was developed by fitting data from a longitudinal study of a selected population of individuals wherein the fitted data comprises levels of said biomarkers and an end point in said selected population of individuals, and wherein said end point is selected from risk for developing cardiovascular disease, the diagnosis of cardiovascular disease, response to cardiovascular disease-modulating drugs, a surrogate cardiovascular disease endpoint, or a complication of cardiovascular disease.

20. The method of claim 1, wherein a model selected from I-III is additionally combined with additional cardiovascular disease markers forming a more complex model, thereby modulating the risk measurement outcome.

21. The method of claim 1, wherein a high LPL mass and low RLP levels as compared to normal control is indicative of a low risk of cardiovascular disease (CVD) in the subject.

22. The method of claim 1, wherein RLP-c values higher than 7.6 mg/dL but lower than 30 mg/dL, RLP-Tg values higher than 50 mg/dL but lower than 70 mg/dL, and/or LPL levels lower than 40 mg/dL are indicative of an intermediate risk of CVD in the subject.

23. The method according of claim 1, wherein RLP-c values higher than 30 mg/dL, RLP-Tg values higher than 70 mg/dL, and/or LPL levels lower than 30 mg/dL are indicative of high risk of CVD in the subject.

24. A diagnostic kit for assessing a cardiovascular disease risk in a subject comprising:
(a) reagents specific for a species of LPL, RLP-c, and/or RLP-Tg;
(b) instructions for use of the reagents to measure a level of the species of LPL, RLP-c, and/or RLP-Tg in a biological sample obtained from the subject;
(c) an information sheet for transforming the measured levels of the species of LPL, RLP-c, and/or RLP-Tg to a ratio and/or logarithmic score and accessing a computer database to compare the score to a reference score and assess the cardiovascular disease risk of the subject based on said comparison.

25. The diagnostic kit of claim 24, wherein the transforming of the measured levels of the species of LPL, RLP-c and RLP-Tg to a ratio and/or logarithmic score is based on any of the following models:
I. ratio score=LPL/RLP-c;
II. ratio score=LPL/RLP-Tg; or
III. logarithmic score=B*log([LPL])−C*log([RLP-c or RLP-tg]), wherein B and C are coefficients calculated from the correlation of values to a population distribution;
wherein an increased, decreased or unchanged score relative to the reference score indicates the level or severity of the cardiovascular disease risk in the subject.

26. The diagnostic kit of claim 25, wherein a score higher than the reference score indicates an increased risk of cardiovascular disease in the subject.

27-46. (canceled)
